(19) United States
(12) Patent Application Publication
(10) Pub. No.: US 2020/0327663 A1
(43) Pub. Date: Oct. 15, 2020

(54) METHOD OF ANALYZING IRIS IMAGE FOR DIAGNOSING DEMENTIA IN ARTIFICIAL INTELLIGENCE

(71) Applicant: HONGBOG Inc., Seoul (KR)

(72) Inventors: Jong NAMGOONG, Seoul (KR); Won-tae CHO, Seoul (KR)

(21) Appl. No.: 16/785,479

(22) Filed: Feb. 7, 2020

(30) Foreign Application Priority Data
Apr. 11, 2019 (KR) 10-2019-0042506

(51) Int. Cl. G06T 7/00; A61B 5/00; G06T 7/11; G06T 3/40

(52) U.S. Cl. CPC G06T 7/0012; A61B 5/4088; A61B 5/7264; G06T 2207/30096; G06T 3/40; G06T 2207/30041; G06T 2207/20084; G06T 7/11

(57) ABSTRACT

A method of analyzing an iris image with artificial intelligence to diagnose dementia in real time with a smart phone according to an embodiment of the present invention includes receiving an input image of a user's eye from user equipment; extracting a region of interest (RoI) from the input image to extract an iris; resizing the extracted RoI to a square shape and scaling the RoI; applying a deep neural network to the resized and scaled RoI; detecting a lesional area by applying detection and segmentation to an image acquired by applying the deep neural network; and diagnosing dementia by determining a position of the lesional area through the detection and by determining a shape of the lesional area through the segmentation.

### CROSS-REFERENCE TO RELATED APPLICATION

This application claims priority to and the benefit of Korean Patent Application Number 10-2019-0042506, filed on Apr. 11, 2019, the entire content of which is incorporated herein by reference.

### BACKGROUND

### 1. Field

The present invention relates to a method of analyzing an iris image with artificial intelligence to diagnose dementia, and more particularly, to a method of predicting diagnoses of a type and even a sign of dementia, designing a lightweight neural network which enables a user to be diagnosed even through a low-performance mobile device, and showing even a position of a corresponding lesional area to improve reliability to a patient with dementia.

### 2. Description of the Related Art

Dementia refers to a state of a human who has difficulties in his or her daily life or social life due to executive and other dysfunctions of the temporal and frontal lobes including memory, attention, linguistic functions, and visuospatial abilities which are some cognitive functions of human. Dementia is classified as Alzheimer's disease, vascular dementia, Lewy body dementia, frontal lobe dementia, etc. according to the causes. Currently, 70% of patients with dementia have Alzheimer's disease.

Alzheimer's disease gradually degrades cognitive abilities and frequently begins with memory loss. Causal clumps composed of abnormal protein aggregation referred to as amyloid plaque and protein referred to as neurofibrillary tangle are tangled with each other and kill cells by blocking communication with neurons such that Alzheimer's disease may develop.

Vascular dementia is a cognitive disorder caused by vascular brain damage. For this reason, an apoplexy patient is likely to suffer from dementia. Further, symptoms of vascular dementia may be similar to those of Alzheimer's disease, and vascular dementia and Alzheimer's disease frequently develop together.

In the case of Lewy body dementia, abnormal protein deposits referred to as alpha-synuclein are generated in a particular brain region. The alpha-synuclein causes changes in movement, thought, and behavior. Also, attention and thinking power are greatly changed. A patient is frequently diagnosed with Parkinson's disease when movement symptoms develop first and is diagnosed with Lewy body dementia when cognitive symptoms develop first.

Frontal lobe dementia is also referred to as frontotemporal dementia and develops when the frontal and temporal lobes are gradually damaged. Damage to the frontal lobe results in behavioral symptoms and personality changes, and damage to the temporal lobes results in a language disorder. Sometimes, the two symptoms develop together.

An iris is an extension of the brain and has hundreds of thousands of nerve endings (autonomic nerves, oculomotor nerves, and sensory nerves), capillaries, and a muscle fiber structure. Therefore, an iris is connected to all organs and tissues through the brain and the nervous system, and it is possible to serve as a direct diagnostic indicator of health of the whole body.

On the basis of this, according to iridology, disease status of relevant tissue is diagnosed from all changes in a patient's iris, and it is found what the body requires. In other words, since irises have the most complex membrane structure and are connected to the cerebra and each part of the body through nerves, information of chemical and physical changes in respective pieces of tissue and organs in the body is transmitted with vibrations and changes the form of fibrous tissue. Using a change in the form of fibrous tissue, it is possible to read and diagnose health status of an individual or status of reaction to treatment, the human skeleton, recovery from a disease, and development of a disease. Therefore, a change in the form of fibrous tissue may become a basis for diagnosing dementia through an iris.

Meanwhile, the general conventional methods of diagnosing dementia are classified into interview tests and laboratory tests, and laboratory tests include brain magnetic resonance imaging (MRI) or computerized tomography (CT) scanning, a blood test, and a cerebrospinal fluid test. An existing artificial intelligence for diagnosing dementia diagnoses dementia using a convolutional neural network (CNN) on the basis of an MRI or CT image. The CNN may include convolutional layers and fully-connected (FC) layers. According to the conventional art, the amount of calculation of a convolutional layer is F²NK²M(F=feature-map, N=input channels, K=kernel, and M=output channels). Therefore, although parallel processing can be rapidly supported due to the development of computer central processing units (CPUs) and the remarkable development of graphics processing unit (GPU) technology, the amount of calculation increases exponentially with an increase in the size of an image. For this reason, it takes a great deal of time to train a CNN which is a deep neural network.

A large amount of calculation is performed in FC layers in a deep neural network according to conventional arts. FC layers lose spatial information which was originally a three-dimensional image by converting features extracted in convolutional layers into one-dimensional vectors. Also, since the most amount of calculation of a deep neural network is performed in FC layers, overfitting may occur, and a processing rate is very slow.

Further, the conventional artificial intelligence for diagnosing dementia is fundamentally based on expensive equipment and data obtained through photography at a specific place. As a result, a user suffers from high cost, experiences inconvenience, and must undergo an invasive method.

Consequently, there is a need for a noninvasive technology for diagnosing dementia at low cost without temporal or spatial limitations. Also, there is a need for a technology for enabling a patient to know a diagnosis result and providing reliability by increasing the accuracy.

### SUMMARY

The present invention is ultimately directed to providing a lightweight neural network which enables a mobile device with less hardware performance than a desktop computer to make a diagnosis in real time, that is, which is concentrated on a processing rate, by solving a problem of a conventional dementia diagnosis apparatus, that is, a low processing rate, and also directed to accurately making a diagnosis with as high accuracy as a desktop computer.

The present invention is also directed to providing reliability to a patient by showing statistical results of dementia diagnoses and visualizing a lesion position.

The present invention is also directed to diagnosing the probability of dementia and the degree of development of the dementia according to detection and analysis results on the basis of big data, which represents the probability of dementia and the degree of development of dementia according to a position and shape of a lesional area, and to notifying a user in real time that an additional test is required if necessary.

Technical objectives of the present invention are not limited to those mentioned above, and other technical objectives not mentioned above will be clearly understood from the following description by those of ordinary skill in the art to which the present invention pertains.

One aspect of the present invention provides a method of analyzing an iris image with artificial intelligence to diagnose dementia in real time with a smart phone, the method including receiving an input image of a user's eye from user equipment; extracting a region of interest (RoI) from the input image to extract an iris; resizing the extracted RoI to a square shape and scaling the RoI; applying a deep neural network to the resized and scaled RoI; detecting a lesional area by applying detection and segmentation to an image acquired by applying the deep neural network; and diagnosing dementia by determining a position of the lesional area through the detection and by determining a shape of the lesional area through the segmentation. The extracting of the RoI further includes extracting the RoI which is a minimum area required to extract an iris by excluding an area not used for dementia diagnosis from the input image. The applying of the deep neural network further includes resizing the extracted RoI in the input image to the square shape and compressing and optimizing pixel information values into one piece of data by normalizing the pixel information values into values between 0 and 1 and converting the normalized pixel information values into bytes. The diagnosing of dementia further includes diagnosing a type of dementia on the basis of the position and shape of the lesional area.

In the method of analyzing an iris image with artificial intelligence, the extracting of the RoI may further include, when the input image is tilted with respect to a vertical direction, aligning the input image by an angle at which the input image is tilted with respect to the vertical direction using a preset virtual axis and then extracting the RoI.

In the method of analyzing an iris image with artificial intelligence, the resizing and scaling of the RoI may include optimizing data of the iris image by resizing the RoI to the square shape, normalizing pixel information values into values between 0 and 1, converting pixel information values into bytes, and compressing the RoI into one piece of data.

In the method of analyzing an iris image with artificial intelligence, the deep neural network may include a convolutional neural network (CNN) to prevent spatial information of the iris image from being lost.

In the method of analyzing an iris image with artificial intelligence, the user equipment may include a camera unit, and the camera unit may include a general mobile camera and an iris recognition camera, or an iris recognition lens may be attached to the camera unit.

In the method of analyzing an iris image with artificial intelligence, the applying of the deep neural network may further include using separable convolution and atrous convolution.

The method of analyzing an iris image with artificial intelligence may further include generating a visualized image, which is a basis for dementia diagnosis, on the basis of the position and shape of the lesional area.

The method of analyzing an iris image with artificial intelligence may further include diagnosing signs of dementia on the basis of the position and shape of the lesional area.

The method of analyzing an iris image with artificial intelligence may further include: accumulating big data representing a probability of dementia and a degree of development of dementia according to a position and shape of a lesional area; determining a probability of dementia and a degree of development of dementia according to the position and shape of the lesional area on the basis of the big data; and notifying the user equipment in real time that an additional test including an interview test and a laboratory test is required according to the probability of dementia and the degree of development of dementia. The type of dementia may include Alzheimer's disease, vascular dementia, Lewy body dementia, and frontal lobe dementia, the probability of dementia may be classified by percentage, and the degree of development of dementia may be classified as an early stage, an intermediate stage, and an end stage.

In the method of analyzing an iris image with artificial intelligence, an activation function and a focal loss method may be used in the CNN.

According to the above-described present invention, it is possible to obtain the following effects. However, effects of the present invention are not limited thereto.

First, the present invention makes it possible to provide reliability to a patient by showing statistical results of dementia diagnoses and also visualizing a lesion position while concentrating on not only accuracy but also a processing rate for diagnosing signs of dementia and making a diagnosis according to a classification. Also, the present invention ultimately enables a person to be diagnosed in real time even with a mobile device with a poor hardware environment through a lightweight neural network concentrated on a processing rate.

Second, the present invention makes it possible to diagnose the probability of dementia and the degree of development of the dementia according to detection and analysis results on the basis of big data, which represents the probability of dementia and the degree of development of dementia according to a position and shape of a lesional area, and to notify a user in real time that an additional test is required by push alarm and the like if necessary.

Technical objectives of the present invention are not limited to those mentioned above, and other technical objectives not mentioned above will be clearly understood from the following description by those of ordinary skill in the art to which the present invention pertains.

### BRIEF DESCRIPTION OF THE DRAWINGS

FIG. 1 is a conceptual diagram of an overall system according to an embodiment of the present invention.

FIG. 2 is a diagram showing a configuration of a system according to an embodiment of the present invention.

FIG. 3 is a flowchart illustrating a method of analyzing an iris image with artificial intelligence to diagnose dementia according to an embodiment of the present invention.

FIG. 4 is a detailed block diagram of a deep neural network learning unit according to an embodiment of the present invention.

FIG. 5 is a diagram showing general convolution and atrous convolution according to an embodiment of the present invention.

FIG. 6 is a diagram showing general convolution and separable convolution according to an embodiment of the present invention.

FIG. 7 is an exemplary diagram showing a lesion position determined by a neural network in a heat map visualization according to an embodiment of the present invention.

FIG. 8 is a block diagram for detecting features which have undergone convolution according to an embodiment of the present invention.

FIG. 9 is a graph of an activation function of a deep neural network according to an embodiment of the present invention.

### DETAILED DESCRIPTION

Hereinafter, exemplary embodiments of the present invention will be described in detail with reference to the accompanying drawings. A detailed description to be disclosed below together with the accompanying drawings is to describe the exemplary embodiments of the present invention, and various modifications and alterations can be made from the embodiments. The detailed description does not represent the sole embodiment for carrying out the present invention.

The embodiments are provided merely to fully disclose the present invention and completely inform those of ordinary skill in the art of the scope of the present invention. The present invention is defined by only the scope of the claims.

In some cases, known structures and devices may be omitted or block diagrams mainly illustrating key functions of the structures and devices may be provided so as to not obscure the concept of the present invention. Throughout the specification, like reference numerals will be used to refer to like elements.

Throughout the specification, when a part is referred to as “comprising” or “including” a component, this indicates that the part may further include another element instead of excluding another element unless particularly stated otherwise.

The term “ . . . unit” used herein refers to a unit that performs at least one function or operation and may be implemented in hardware, software, or a combination thereof. Further, “a” or “an,” “one,” and the like may be used to include both the singular form and the plural form unless indicated otherwise in the context of the present invention or clearly denied in the context.

In addition, specific terms used in the embodiments of the present invention are provided only to aid in understanding of the present invention. Unless otherwise defined, all terms including technical and scientific terms used herein have the same meaning as commonly understood by those of ordinary skill in the art to which the present invention pertains. The use of the specific terms may be modified in a different form without departing from the technical spirit of the present invention.

Hereinafter, exemplary embodiments of the present invention will be described in detail with reference to the accompanying drawings. A detailed description to be disclosed below together with the accompanying drawings is to describe the exemplary embodiments of the present invention and does not represent the sole embodiment for carrying out the present invention.

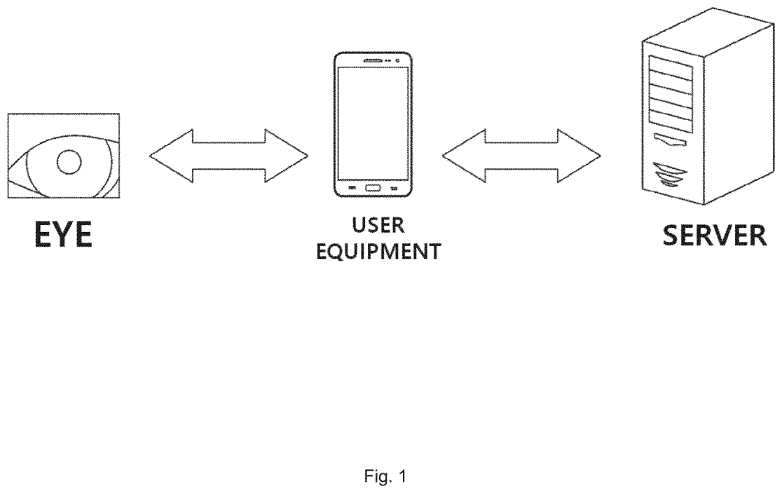

FIG. 1 is a conceptual diagram of an overall system according to an embodiment of the present invention.

Referring to FIG. 1, user equipment may be an electronic device including a smart phone, a tablet personal computer (PC), a laptop computer, or the like. The user equipment may include a camera, and the camera may include an iris recognition camera, or an iris recognition lens may be attached to the camera. Therefore, when a user's eye is photographed using the user equipment, it is possible to acquire a high-resolution screen or image of a specific part including an iris. The image acquired by photographing the user's eye through the user equipment is input to a server and used for dementia diagnosis and the like. In connection with dementia diagnosis, an exemplary embodiment of the present invention will be described in further detail below with reference to the accompanying drawings.

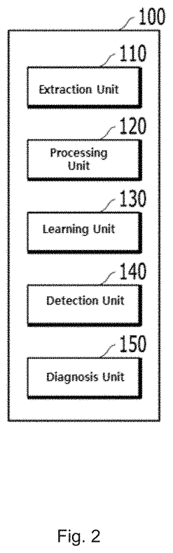

FIG. 2 is a diagram showing a configuration of a system according to an embodiment of the present invention.

Referring to FIG. 2, the system according to the embodiment of the present invention may include an extraction unit 110 which extracts a region of interest (RoI) to extract an iris from an input image, a preprocessing unit 120 which resizes the extracted RoI to a polygonal shape, that is, a square shape of various sizes, and scales the RoI, a learning unit 130 which applies a deep neural network to the resized and scaled RoI, a detection unit 140 which detects a lesional area by applying detection and segmentation to an image acquired by applying the deep neural network, and a diagnosis unit 150 which diagnoses dementia by determining a position of the lesional area through the detection and determining a shape of the lesional area through the segmentation.

The extraction unit 110 may acquire an image from even a low-performance mobile device.

The extraction unit 110 may acquire images which will be used to diagnose dementia and then remove an image part unnecessary for diagnosis to increase a processing rate. According to an embodiment, the iris images may include tilted images. Therefore, it is possible to align the tilted images straight up and down using a preset virtual axis and then extract only RoIs from the eye images. An RoI refers to a minimum area for extracting an iris required for dementia diagnosis excluding an unnecessary area.

The reason that an RoI should be extracted is to reduce the amount of calculation as much as possible for lightweighting by removing an area unnecessary for dementia diagnosis. After an RoI is extracted, an unnecessary area may be colored in grey, and the RoI may be transmitted to the preprocessing unit 120.

The preprocessing unit 120 may resize the iris image obtained from the extraction unit 110 to a square (N×N) size. When an image increases in size, the amount of calculation is multiplied by the increment. Therefore, the image is adjusted to an appropriate size for lightweight artificial intelligence, and pixel information values of 0 to 255 are normalized into values between 0 and 1 so that the values may not have errors or may not deviate from expected values.

In the case of a general neural network, it is necessary to planarize an iris image, which is three-dimensional data, into one dimensional image, and thus spatial information of the image is lost during the process. Therefore, the learning unit 130 may include a convolutional neural network (CNN) which can learn an iris image while maintaining spatial information of the iris image.

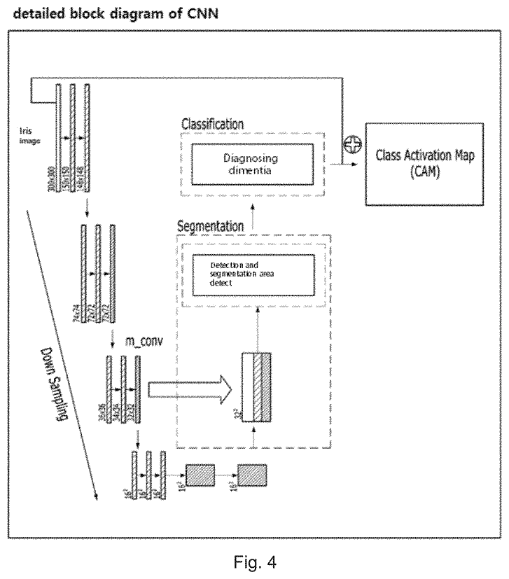

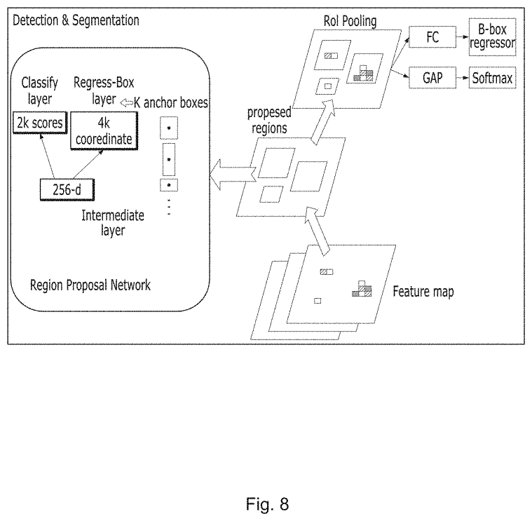

The learning unit 130 and the detection unit 140 will be described in detail with reference to FIGS. 4 and 8, respectively.

The detection unit 140 detects a detailed position and shape of a lesional area on the basis of overall iris characteristics extracted through the learning unit 130, and the diagnosis unit 150 analyzes and classifies dementia respectively on the basis of segmentation and detection so that a type of dementia may be determined.

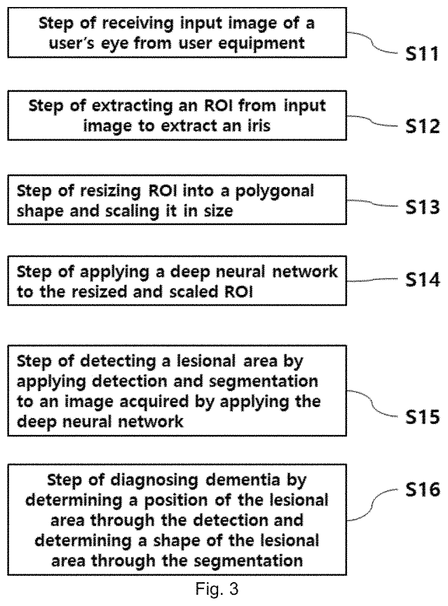

FIG. 3 is a flowchart illustrating a method of analyzing an iris image with artificial intelligence to diagnose dementia according to an embodiment of the present invention.

Referring to FIG. 3, an input image of a user's eye may be received from user equipment (S11), and an RoI may be extracted from the input image to extract an iris (S12). The user equipment may include an iris recognition camera or employ a camera equipped with an iris recognition lens. Therefore, it is possible to readily extract an RoI including an iris with a higher resolution than that of a general camera.

The extracted RoI may be resized into a polygonal shape and scaled in size (S13), and a deep neural network may be applied to the resized and scaled RoI (S14). The “various sizes” may include a reduction, increase, etc. in image size while the square shape is maintained. When the deep neural network is applied, only an RoI may be extracted by coloring an area, which is not extracted as the RoI, in grey because the area is an unnecessary area. Accordingly, an unnecessary amount of calculation may be reduced, which may be a basis for a lightweight neural network concentrated on a processing rate.

Further, a lesional area may be detected by applying detection and segmentation to an image acquired by applying the deep neural network (S15). It is possible to diagnose dementia by determining a position of the lesional area through the detection and determining a shape of the lesional area through the segmentation (S16). A type of dementia may be determined on the basis of the position and shape of the lesional area. The types of dementia include Alzheimer's disease, vascular dementia, Lewy body dementia, and frontal lobe dementia by way of example. When the probability of dementia is determined on the basis of the position and shape of the lesional area, for example, a higher color strength of the lesional area may represent the higher probability of dementia by percentage and the like. Further, the degree of development of dementia may be represented on the basis of the position and shape of the lesional area. For example, the degree of development of dementia may be classified and represented as an early stage, an intermediate stage, or an end stage.

FIG. 4 is a detailed block diagram of the learning unit 130 according to an embodiment of the present invention.

Referring to FIG. 4, an iris image processed through the preprocessing unit 120 is received as an input and may be modified to various sizes such as 300×300, 500×500, and 400×400.

Key characteristics of a deep neural network include, first, convolutional layers generating feature maps by applying various filters to an input image. In other words, convolutional layers serve as templates which extract features of a high-dimensional input image. Second, downsampling refers to a neuron layer which reduces a spatial resolution of a generated feature map. Third, an activation function refers to a role for receiving an input signal, generating an output signal in response to the input signal when the input signal satisfies a specific threshold value, and transmitting the output signal to the next layer. This is modeled after neuroscience reporting that a neuron transmits a signal to the next neuron when a strong stimulus (=a specific threshold value) is received. In general, a rectified linear unit (ReLU) function is used and is represented as y=max(0, x).

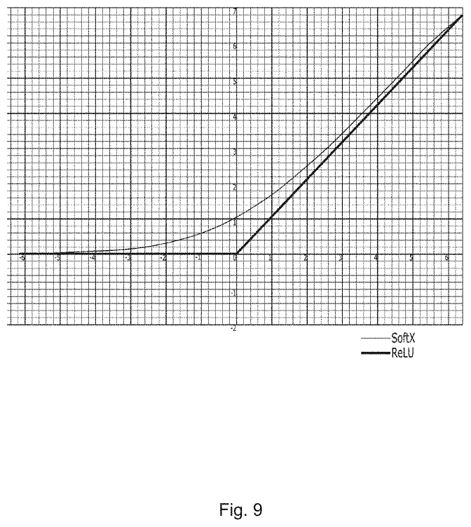

However, the ReLU has a demerit that when x of an input signal is 0 or less, the signal is transmitted with a value of 0, that is, all signals of negative values are ignored. To complement the ReLU, according to the present invention, the function is corrected into y=1/(1−exp?(x))*x so that even a signal having x of 0 or less may be transmitted with a certain degree of stimulus. The corrected function is referred to as softX and will be described in further detail with reference to FIG. 9.

When the aforementioned layers are consecutively stacked and extracting local features from the image begins with a frontend layer and reaches to a backend layer, only a global feature which represents the overall image remains.

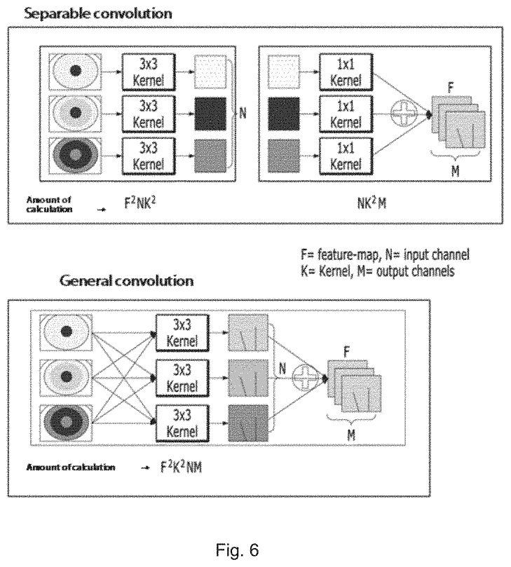

The amount of calculation of each general convolutional layer is F2NK2M (F=feature-map, N=input channels, K=kernel, and M=output channels). However, according to the present invention, the amount of calculation of a convolutional layer is reduced by separating the expression into F2NK2+F2MN, which will be described in detail with reference to FIG. 6.

Meanwhile, there are factorization methods as a method for reducing the amount of calculation as described above. According to a factorization method, a 5×5 filter is factorized into 1×5+5×1 filters. In this case, the amount of calculation may be reduced by a ratio of 25:10, that is, about ½ to ⅓. Assuming that an input image is 7×7, a 1×1 value is output when a 7×7 filter is used. On the other hand, when a 3×3 filter is used, a 5×5 value is output. When a 3×3 filter is used again, a 3×3 value is output, and when a 3×3 filter is used again, a 1×1 value is output. As a result, to obtain a 1×1 value as an output, using one 7×7 filter is equivalent to use of three 3×3 filters. In terms of the amount of calculation, 49:9+9+9=49:27, and thus it is possible to reduce the amount of calculation by about 45%. On the basis of this idea, a combination of a separable convolution method and a factorization method is used in the present invention.

Meanwhile, in the operation process of the learning unit 130, a lesional area has little pixel information compared to an overall iris image. Therefore, it may be difficult to extract a feature of a lesional area having a local feature due to the characteristic of a general deep neural network having a global feature representing an overall image when many layers overlap.

According to the present invention, however, during training of a deep neural network, it is possible to change a loss function into a specific loss function by giving a greater weight to a local feature than a global feature.

Here, a loss function refers to a process in which a deep neural network calculates an error between an answer predicted with a feature extracted by a last convolutional layer and a correct answer and updates a weight by backpropagating a variation of the error. Repeating this process is referred to as training a neural network.

In this regard, while a loss function which is generally used to train a neural network frequently employs cross-entropy (CE) loss, the present invention employs a focal loss method as the aforementioned specific loss function.

Specifically, when a deep neural network extracts feature maps, it is easier to extract a global feature than a local feature as described above. Therefore, during training of a deep neural network, an area (a global feature) other than a lesional area (a local feature), which is to be detected, is learned more than the lesional area.

However, according to the present invention, it is necessary to learn a feature of a very small lesional area better than a feature of an overall iris image. To this end, a focal loss method is used.

A CE function which is a generally used loss function is CE( )=−log( ), and the focal loss function used in the present invention is defined as F( )=−log( )(probability of correct answer).

In brief, in the focal loss function used in the present invention, indicates the probability of a correct answer, and thus (1−) indicates the probability of an incorrect answer.

Therefore, an overall value of the loss function is reduced with an increase in “probability of correct answer” and is increased with a reduction therein.

Also, when data is biased to a specific class during the training, it is difficult to learn features of classes having little data, and thus a correct answer rate is low. In this case, it is possible to use a focal loss method.

The size of a lesion to be detected in an iris image is very small as compared to the overall image. For this reason, in the case of classifying the extracted lesional area, training is performed with less data than that of the overall image. Therefore, training is not performed appropriately, and accuracy is low.

In this regard, it is possible to obtain high accuracy using the focal loss method proposed in the present invention.

Meanwhile, details of a feature are supplemented by upsampling an image of a feature map extracted from a last convolutional layer to a double size image and combining the double size image with a feature map of a layer of m_conv (see FIG. 4).

The feature map whose details have been supplemented is input to the detection unit 140, which detects detection and segmentation areas, and will be described in detail with reference to FIG. 8.

Finally, a weight learned on the basis of the position and shape of the lesional area detected by the detection unit 140 is loaded to make a calculation (;=class activation map (CAM), w=weight, =unit of activation function). Then, the deep neural network determines a position and shape of the lesional area and generates a visualized image indicating whether the user has been diagnosed with dementia.

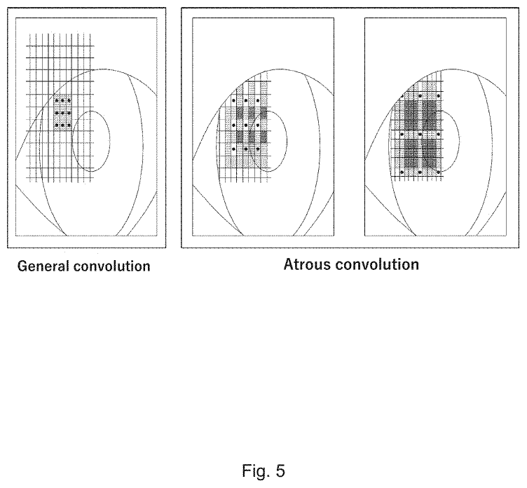

Referring to FIG. 5, like general convolution, but unlike convolution performed using only pixels at positions of a circular dot, atrous convolution may enlarge a receptive field colored in grey without resolution loss, but the number of dots on which convolution is performed remains as it is. Therefore, it is possible to draw information from various scales.

In other words, when downsampling is performed in a pooling layer, a spatial resolution is reduced, and an image more indistinct than the original image is obtained. However, when the atrous convolution is used, it is possible to extract a high-resolution image similar to the original image, and the atrous convolution may replace a pooling layer.

Referring to FIG. 6, a three-dimensional image is composed of red, green, and blue (RGB). Therefore, during training of a neural network, one kernel (hereinafter, also referred to as “filter”) calculates R, G, and B values in a duplicate manner to extract a feature in general convolution.

As a result, due to the characteristic of convolution of extracting a feature through a filter and generating several features, even different filters calculate the same R, G, and B values and may extract identical features. Therefore, it may be difficult to extract various features.

On the other hand, in separable convolution, R, G, and B values are separately calculated, that is, filters are separately generated for R, G, and B values. Therefore, a color feature may be extracted in further detail, and then it is possible to extract various features.

Also, general convolution has the amount of calculation of F2NK2M because the calculation is performed at once and a feature is extracted. However, in separable convolution, calculation of R, G, and B values is separated from generation of a filter for extracting a feature through the calculation, and the amount of calculation is F2NK2+F2MN. Therefore, it is possible to increase a processing rate to eight to nine times a conventional processing rate.

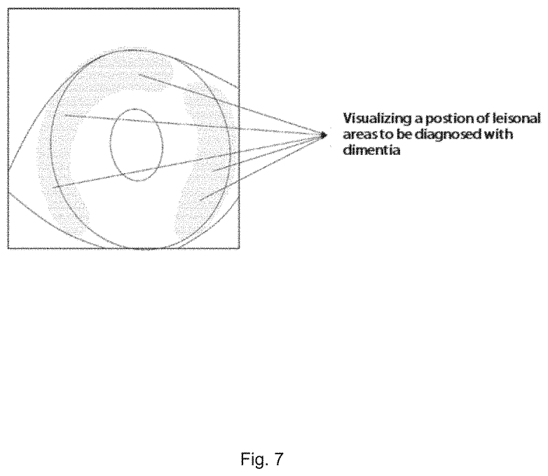

Referring to FIG. 7, thicker grey color shows from where a neural network has mainly extracted features in a CAM and on the basis of which area a diagnosis has been made.

Specifically, assuming that fully-connected (FC) layers are replaced with global average polling (GAP) and there are two 16×16 feature maps (=16×16×2) by way of example, the feature maps are mapped to 1×1×256 vectors or so on. In other words, all the feature maps are pooled and mapped to several neurons. As a result, in the above example, the feature maps are mapped to 256 neurons.

Therefore, each feature map becomes several neurons through GAP, and the neurons are given appropriate weights and classified. The weights are used to generate a CAM with the neurons overlapping the original iris image. As a result, when a weight increases, grey becomes darker in a CAM. This is a major basis for classifying dementia.

FIG. 8 is a diagram of the detection unit 140 of FIG. 2 and is also a diagram showing detection and segmentation area detection of FIG. 4.

Referring to FIG. 8, a position and shape of a lesional area are detected using feature maps of layers of the final stage of FIG. 4.

A region proposal network (RPN) is used to extract a lesional area for dementia diagnosis from a feature map. In the network, candidate ROIs in which an object may exist are detected through a preset anchor box first, and the candidate RoIs are classified according to objects by a classifier.

Since the extracted RoIs have different sizes, it is difficult to process the extracted RoIs in a general deep neural network which requires a fixed size. Therefore, the RoIs of different sizes are converted to the same size through an RoI pooling layer.

The size of converted RoIs in which an object has been detected is segmented in units of pixels. However, according to a conventional method, alignment is not taken into consideration.

In other words, even when the size to which conversion has been performed in an RoI pooling layer, that is, a detected size of an object, has a value below the decimal point, the value is removed by rounding the size to the nearest 1. Therefore, the object is in a poor alignment state.

Also, pixel units are calculated through FC layers. As described above regarding FC layers, FC layers lose spatial information of an original three-dimensional image by converting features extracted in convolution layers into one-dimensional vectors. Therefore, accuracy is degraded.

On the other hand, according to the present invention, the decimal point is kept intact, and bilinear interpolation is used for accurate alignment. Therefore, according to the present invention, it is possible to know the position and shape of a lesional area.

Further, according to the present invention, FC layers are changed to 1×1 convolution layers to solve the problem of FC layers. FC layers are named “fully-connected” in the meaning of connecting all neuron layers and calculating correlations. Since 1×1 convolution is performed through a 1×1 filter, correlations of each pixel may be calculated, and also spatial information may be maintained. Therefore, it is possible to increase accuracy.

Finally, the probability of dementia is estimated using a weight obtained by training the deep neural network on the basis of the position and shape of the lesional area extracted through segmentation and detection.

Referring to FIG. 9, an activation function is shown as described above in the description of key characteristics of a deep neural network.

As described above, an activation function refers to a role for receiving an input signal, generating an output signal in response to the input signal when the input signal satisfies a specific threshold value, and transmitting the output signal to the next layer. Generally, the ReLU function is used.

However, since the ReLU has a demerit that when x of an input signal is 0 or less, the signal is transmitted with a value of 0, learning is not performed smoothly by the deep neural network learning unit 130. The reason is as follows. When a deep neural network has deep layers, it is possible to extract detailed features. Meanwhile, the neural network calculates an error due to a loss function and learns the error by backpropagating the error. The error is calculated with a differential value, that is, a variation. When an error is backpropagated while being multiplied by a differential value in each layer, a variation becomes very small going toward a frontend layer and converges without being transmitted. When this is applied to a ReLU, all values of 0 or less are processed as 0. As a result, 0 is obtained by differentiating 0. For this reason, learning is not smoothly performed due to the characteristic of a deep neural network performing learning while a weight is being updated.

To solve this problem, even values of 0 or less can be learned according to the present invention. Therefore, learning can be smoothly performed, and accuracy can be increased accordingly.

In addition to the above-described embodiment, according to the present invention, it is possible to store big data, which represents the probability of dementia and the degree of development of dementia according to a position and shape of a lesional area, to learn and determine the probability of dementia and the degree of development of dementia according to a position and shape of a lesional area on the basis of the big data and to notify user equipment in real time that an additional test including an interview test and a laboratory test is required according to the probability of dementia and the degree of development of dementia. Also, when the degree of development of dementia has been drastically increased over time recently, it is possible to notify the user equipment of such development in real time. The real-time notification through the user equipment may be a popup, a push alarm, and the like.

Meanwhile, the above-described method can be written as a program executable in a computer and may be implemented by a general-use digital computer which runs the program using a computer-readable medium. The structure of data used in the above-described method may be recorded in the computer-readable medium in several ways. The computer-readable medium which stores executable computer code for executing various methods of the present invention includes storage media, such as magnetic storage media (e.g., a read-only memory (ROM), a floppy disk, a hard disk, etc.,) and optical reading media (e.g., a compact disc (CD)-ROM, a digital versatile disc (DVD), etc.).

Those of ordinary skill in the technical field related to embodiments of the present invention will appreciate that the present invention may be implemented in modified forms without departing from the essential characteristics of this disclosure. Therefore, the disclosed methods should be considered from a descriptive point of view rather than a limiting point of view. The scope of the present invention is disclosed not in the detailed description of the present invention but in the claims, and all differences lying within the range of equivalents should be interpreted as being included in the scope of the present invention.

1. A method of analyzing an iris image with artificial intelligence to diagnose dementia in real time with a smart phone, the method comprising:
receiving by the server of an input image of a user's eye from user equipment;
extracting a region of interest (RoI) by the server from the input image to extract an iris;
resizing the extracted RoI to a square shape and scaling the RoI by the server;
applying a deep neural network by the server to the resized and scaled RoI;
detecting a lesional area by the server by applying detection and segmentation to an image acquired by applying the deep neural network; and
diagnosing dementia by the server by determining a position of the lesional area through the detection and by determining a shape of the lesional area through the segmentation,
wherein the extracting of the RoI further comprises extracting the RoI which is a minimum area required to extract an iris by excluding an area not used for diagnosing dementia from the input image,
wherein the applying of the deep neural network further comprises resizing the extracted RoI in the input image to a square shape and compressing and optimizing pixel information values into one piece of data by normalizing the pixel information values into values between 0 and 1 and converting the normalized pixel information values into bytes, and
wherein the diagnosing of dementia further comprises diagnosing a type of dementia based on the position and shape of the lesional area,
wherein the diagnosing the type of dementia based on the position and shape of the lesional area comprises:
accumulating bid data representing a probability of dementia and a degree of
development of dementia according to a position and shape of a lesional area;
determining a probability of dementia and a degree of development of dementia according to the position and shape of the lesional area based on the big data; and
notifying the user equipment in real time that an additional test including an interview test and a laboratory test is required according to the probability of dementia and the degree of development of dementia,
wherein the type of dementia includes Alzheimer's disease, vascular dementia, Lewy body dementia, and frontal lobe dementia,
wherein the probability of dementia is classified by percentage, and
wherein the degree of development of dementia is classified as an early stage, an intermediate stage, and an end stage.

2. The method of claim 1, wherein the extracting of the RoI further comprises, when the input image is tilted with respect to a vertical direction, aligning the input image by an angle at which the input image is tilted with respect to the vertical direction using a preset virtual axis and then extracting the RoI.

3. The method of claim 1, wherein the resizing and scaling of the RoI comprises optimizing data of the iris image by resizing the RoI to the square shape, normalizing pixel information values into values between 0 and 1, converting the pixel information values into bytes, and compressing the RoI into one piece of data.

4. The method of claim 1, wherein the deep neural network includes a convolutional neural network (CNN) to prevent spatial information of the iris image from being lost.

5. The method of claim 1, wherein the user equipment includes a camera unit, and
the camera unit includes a general mobile camera and an iris recognition camera, or an iris recognition lens is attached to the camera unit.

6. The method of claim 4, wherein the applying of the deep neural network further comprises using separable convolution and atrous convolution.

7. The method of claim 1, further comprising generating a visualized image, which is a basis for dementia diagnosis, based on the position and shape of the lesional area.

8. The method of claim 1, further comprising diagnosing signs of dementia based on the position and shape of the lesional area.

9. (canceled)

10. The method of claim 4, wherein an activation function and a focal loss method are used in the CNN.
